(19) United States
(12) Patent Application Publication
(10) Pub. No.: US 2024/0156213 A1
(43) Pub. Date: May 16, 2024

(54) Adjustable Insoles

(71) Applicant: Actics Medical Ltd, Rehovot (IL)

(72) Inventor: Ron Machanian, Rehovot (IL)

(21) Appl. No.: 18/282,575

(22) PCT Filed: Mar. 29, 2022
(86) PCT No.: PCT/IL2022/050337

§ 371 Date: Sep. 18, 2023

Related U.S. Application Data
(60) Provisional application No. 63/167,663, filed on Mar. 30, 2021.

(51) Int. Cl. A43B 17/00; A43B 17/14

(52) U.S. Cl. CPC A43B 17/006; A43B 17/14

(57) ABSTRACT

Presented herein is an insole comprised of: a base layer located at the bottom of the insole; an array of holes that pass through the base layer; a distribution layer that is designed to distribute forces across the area of the insole located above the base layer; a foam layer located above the distribution layer; a sensor layer located above the foam layer; and a fabric layer located at the top of insole. The insole comprises height adjustment components configured to be inserted into and locked in place in any one of the holes in the base layer in order to raise or lower the height of the insole at the location of the hole. Output from the sensor layer provides real-time measurements of physical parameters inside a patients shoe that are analyzed in a system that provides advise to the patient or a foot care specialist for manually adjusting the height adjustment components.

### FIELD OF THE INVENTION

The invention is in the field of orthotics. More particularly, the invention relates to adjustable insoles.

### BACKGROUND OF THE INVENTION

Insoles are often present in footwear produced commercially and/or sold separately to add to shoes. The typical insole is a generic device made of foam rubber and/or cloth.

There are also commercially available unchanging “off the shelf” insoles featuring foam and/or gel and/or air cushions that purport to increase comfort if they are installed in shoes sold separately from the insole. These fixed passive insoles are generic devices that can't be adjusted to the user's personal anatomy or medical condition.

In addition, orthopedists, podiatrists, physiotherapists and other health professionals employ a variety of different personally customized fixed passive insole types for diagnosis, planning and treatment to address various symptoms, usually having to do with pain and discomfort of the feet and/or lower extremity. Conditions such as arthritis, back pain, foot deformities, Plantar fasciitis, heel injuries, Achilles injuries and ruptures, bone spurs, bunions, bursitis, pronation (flat feet), high arch, fractures, hammer toes, sarcopenia, balance impairments, elderly falls, foot inflammation, neuroma, lesions, osteoporosis, diabetic foot ulcers and tendonitis all cause pain. Sufferers from these maladies cannot be treated with generic insoles, since they require a solution that fits their personal foot structure and/or condition.

Treatment planning in medical fields (e.g., orthopedics) or sport training is typically based on professional assessment, manual and/or digital. The assessment can include, besides a physical observation, video and/or computer monitoring of certain actions, in a static or dynamic manner such as standing, running or walking (e.g. in a gait lab). Alternatively, or additionally, the assessment can include evaluation of biomechanical functions, the mobility of the foot and/or ankle and/or muscles and knees and/or hips. Muscles are typically evaluated with respect to tone and/or range of motion. Alternatively, or additionally, range of motion of each joint is evaluated.

Capturing reliable information about a person's gait in real-life environments is challenging. Standard gait-analysis technologies, such as camera-based motion-capture systems and force plates, are expensive and can only be used inside laboratories, so they offer few insights into a subject's biomechanical motion in the real world. Based on the assessment, a treatment plan, including orthopedic shoes and/or customized insoles and/or orthotics (braces), may be proposed.

Existing custom-made insoles typically have a fixed configuration with limited possibility of adjustment. Custom orthotics can be specially designed to match your foot type, foot condition and activity level and are made by an orthotist or other healthcare professional. That requires repetitive visits of the patient to adjust the insoles. Therefore, this process is time consuming and price of insoles is high. As a result, if a person's gait changes (e.g., as a result of weight change, injury or physical activity) or if person replaces his shoes or if a diabetic person needs to offload foot pressure in order to prevent ulceration, the insole cannot over-go a corresponding change nor to fit well in different kind of shoes.

In light of the impending diabetes epidemic and high prevalence of Diabetic Foot Ulceration (DFU) and its associated complications, the need for enhanced prevention of DFUs is clear. At least 70% of amputations are potentially preventable using interventions such as foot care, footwear, custom made insoles, daily monitoring plantar condition, and medical management. However, effective technology is still missing to facilitate monitoring DFU related risk factors on a daily basis, empower patients in self-care as well as engaging them to use these technologies, and effectively coordinate care among circles of care providers and caregivers.

It is therefore a purpose of the present invention to provide orthotic insoles that are configured to be adjusted.

It is another purpose of the present invention to provide orthotic insoles that are configured to provide real-time measurements and/or analysis of physical parameters inside a patient's shoe.

Further purposes and advantages of this invention will appear as the description proceeds.

### SUMMARY OF THE INVENTION

A first aspect of the invention relates to an insole comprised of the following layers:
a) a base layer comprising an array of holes that pass through the base layer, the base layer located at the bottom of the insole;
b) a distribution layer that is designed to distribute forces across the area of the insole, the distribution layer located above the base layer;
c) a foam layer, the foam layer located above the distribution layer;
d) a sensor layer, the sensor layer located above the foam layer; and
e) a fabric layer, the fabric layer located at the top of insole.

Embodiments of the insole comprise height adjustment components configured to be inserted into and locked in place in any one of the holes in the base layer, wherein the height adjustment components are configured to raise or lower the height of the insole at the location of the hole.

In embodiments of the insole the bottom side of the base layer comprises a socket into which electronics and a battery are inserted.

In embodiments of the insole the distribution layer comprises an array of holes that pass through the distribution layer and that corresponds to the array of holes in the base layer.

In embodiments of the insole locations requiring height adjustment are determined using various sensors that are located on the sensor layer.

In embodiments of the insole the sensor layer is a flexible printed circuit board that comprises at least one of each of at least one of the following: pressure sensors, temperature sensors; humidity sensors; leveling sensors; compass sensor; gyroscopes; accelerometers; and antennas.

In a first embodiment of the insole the height adjustment components are rods of varying heights. In these embodiments the height adjustment rods are firmly attached to the base layer by a bayonet connection comprised of pins located near the bottom of each rod that are configured to fit into slots near the top of each hole that passes through rigid material of the base layer.

In a second embodiment of the insole the holes in the base layer of the insole comprise at least one channel composed of two vertical sections and one horizontal section created in the walls of the hole; wherein, the first vertical section has a first end at the bottom of the insole and a second end ending at a first end of the horizontal section and the second vertical section has a first end beginning at a second end of the horizontal section and ending at the top of the insole.

In embodiments of the second embodiment of the insole the height adjustment components are composed of at least one disk comprising: at least one disk lock tooth configured to lock the disk to the base layer of the insole; recesses configured to mate with a tool designed to add the disk to a disk below it or to release the disk from a disk above it; a nipple having a polygon cross section at the top of the disk; a socket having a hexagonal cross section in the bottom of the disk; and a hole passing through the disk.

In these embodiments, the disk is configured to be locked to the base layer by inserting the disk from the bottom of the insole with the nipple upwards and the lock teeth aligned with the first end of the first vertical sections of the channels, pushing the disk upwards until the lock teeth reach the second end of the first vertical section of the channel, and rotating the disk clockwise until the lock teeth enter the horizontal section of the channels. In these embodiments the disk is configured to be unlocked and removed from the base layer in one of two ways: i) by rotating the disk counterclockwise until the lock teeth reach the first end of the horizontal section of the channel; pulling the disk downward in the first vertical section of the channel until the lock teeth exit the first vertical section of the channel and removing the disk from the bottom of the base layer; and ii) by rotating the disk clockwise until the lock teeth reach the second end of the horizontal section of the channel; pushing the disk upward in the second vertical section of the channel until the lock teeth exit the second vertical section of the channel and removing the disk from the top of the base layer.

In embodiments of the second embodiment of the insole the height adjustment component is a stack of at least two of the disks formed by inserting the nipple at the top of a bottom disk into the socket in the bottom of the disk above it and repeating the process adding as many disks as necessary to attain the required height.

A second aspect of the invention relates to a dedicated tool configured to insert a height adjustment component comprised of a disk or a stack of disks into the second embodiment of the insole.

Embodiments of the tool comprise: an elongated cylindrical barrel, which contains in its interior a stack of disks; a lock button located on the base of the barrel; a dial located near the bottom of the barrel; a lock shaft that passes through the holes in the disks; an expansion element attached at the top end of the lock shaft; and an activation shaft, which passes through the interiors of the lock shaft and the expansion element.

A third aspect of the invention is a method of using the dedicated tool of the second aspect, to add at least one disk to a height adjustment component comprised of a disk or a stack of disks in the second embodiment of the insole, the method comprising:
a) locating the tool at a required hole on the underside of the base layer of the insole;
b) locking the tool in place by inserting the lock shaft through the hole in the base layer and the expansion element fully into a corresponding hole in the distribution layer of the insole and pressing the lock button on the tool;
c) preparing one disk by turning the dial of the tool clockwise one click;
d) turning the tool clockwise until one disk clicks into place;
e) repeating steps c and d as often as necessary to add more disks; and
f) pushing the lock button on the tool to release the tool from the distribution layer and remove the tool from the insole leaving the last disk added in locked in the base layer.

A fourth aspect of the invention is a method of using the dedicated tool of the second aspect to remove at least one disk from a height adjustment component comprised of a disk or a stack of disks in the second embodiment

of the insole, the method comprising:
a) locating the tool at the point required on the underside of the insole;
b) locking the tool in place by inserting the shaft through the hole in the base layer and the expansion element fully into a corresponding hole in the distribution layer of the insole and pressing the lock button on the tool;
c) preparing one disk by turning the dial of the tool counter-clockwise one click;
d) turning the tool counter-clockwise until one disk clicks out of the hole in the base layer;
e) repeating steps c and d to remove more disks as necessary; and
f) pushing the button on the tool to release and remove the tool from the insole.

A fifth aspect of the invention is a system configured for adjusting the height of an insole at specific locations on the insole, the system comprising:
a) at least one insole according to the first aspect comprising height adjustment components;
b) a data processor configured to receive at least one output signal from sensors in the sensor layer of each insole and to translate the output signals to user advice;
d) a user interface on which the user advice is displayed;
wherein the user advice relates to adjustment of the height of at least one of the height adjustment components in each of the insoles.

All the above and other characteristics and advantages of the invention will be further understood through the following illustrative and non-limitative description of embodiments thereof, with reference to the appended drawings.

### BRIEF DESCRIPTION OF THE DRAWINGS

FIG. 1A is a schematic exploded view of layers of an insole illustrating incorporation of height adjustment mechanisms;

FIG. 1B schematically shows a top view of a plurality of holes into which height adjustment mechanisms are installed in a base plate layer of the insole of FIG. 1A;

FIG. 1C schematically shows a top perspective view of an embodiment of an insole illustrating additional features of the base plate layer of the insole illustrated in FIGS. 1A and 1B;

FIG. 2A is a transverse cross section of an exemplary height adjustment mechanism using a screw;

FIG. 2B is a side view of a portion of the exemplary height adjustment mechanism of FIG. 2A;

FIG. 2C is a bottom view of a portion of the exemplary height adjustment mechanism of FIG. 2A showing a manual adjustment interface;

FIG. 2D is a transverse cross section of an exemplary diagonal joint rotary lift height adjustment mechanism in a closed (retracted) operational state;

FIG. 2E is a bottom view of a portion of the exemplary height adjustment mechanism of FIG. 2D showing a manual adjustment interface;

FIG. 2F is a transverse cross section of the height adjustment mechanism of FIG. 2D in an open (extended) operational state;

FIG. 2G is a side view of an exemplary bayonet mount height adjustment mechanism;

FIG. 2H is a side view of the bayonet mount height adjustment mechanism of FIG. 2G rotated 90° with respect to FIG. 2G;

FIG. 2I is a bottom view of a portion of the exemplary height adjustment mechanism of FIGS. 2G and 2H showing a manual adjustment interface;

FIG. 3A is a schematic exploded view of an embodiment of an insole;

FIG. 3B symbolically shows an exploded view of another embodiment of an insole;

FIG. 4 schematically shows an exemplary embodiment of a sensor layer in the insoles of FIGS. 3A and 3B;

FIG. 5 schematically shows the three dimensional shape of a base layer in the insole of FIGS. 3A and 3B;

FIG. 6 is a bottom view of the base layer of FIG. 5 showing an array of holes through the base layer;

FIG. 7A shows a set of height adjustment components in the form of rods for use in the insole of FIG. 3B;

FIG. 7B symbolically shows a number of holes in an insole without any height adjustment components installed in them;

FIG. 7C symbolically shows the holes in FIG. 7B with height adjustment rods inserted in them;

FIG. 8A schematically shows a top view of an embodiment of a disk that will form a component of a height adjustment component in the insole of FIG. 3A;

FIG. 8B shows a side view of a single disk of FIG. 8A and a stack of seven disks of the disks;

FIG. 8C symbolically shows the influence of height adjustment components comprised of stacks of different numbers of disks of FIG. 8A located in holes in the base layer on the contour of the distribution layer of insole of FIG. 3A;

FIG. 9 schematically shows a cross-sectional view of the upper part of an empty hole in the base layer that has been configured for the insertion of disks;

FIGS. 10A, 10B, and 10C schematically show a tool configured to insert disks of FIG. 8A through a hole in a base layer of an insole of FIG. 3A;

FIGS. 11A and 11B schematically show respectively how an expansion element of a tool of FIGS. 10A and 10B is locked and unlocked in a hole of the distribution layer of the insole of FIG. 3A;

FIGS. 12 and 13 schematically respectively illustrate the processes of using the tool of FIGS. 10A, 10B, and 10C to add and remove disks of FIG. 8A to/from a stack of disks;

FIG. 14 is a schematic representation of a data acquisition layer of an insole;

FIG. 15 is a schematic representation of a system for providing user advice for adjusting the height adjustment components in an insole; and

FIG. 16 schematically shows an example of user advice displayed on a smart device.

### DETAILED DESCRIPTION OF EMBODIMENTS

Embodiments of the invention relate to adjustable insoles that can be used to custom fit insoles to different/changing physiological conditions and status of users and/or to different activities of a same user. Embodiments of the invention can be used to diagnose and or detect and/or warn a user of a nascent foot ulcer and/or other foot disorders and to initiate and enable correction and/or adjustment and/or treatment plans that will be performed manually.

The principles and operation of adjustable insoles and/or systems according to exemplary embodiments of the invention may be better understood with reference to the drawings and accompanying descriptions.

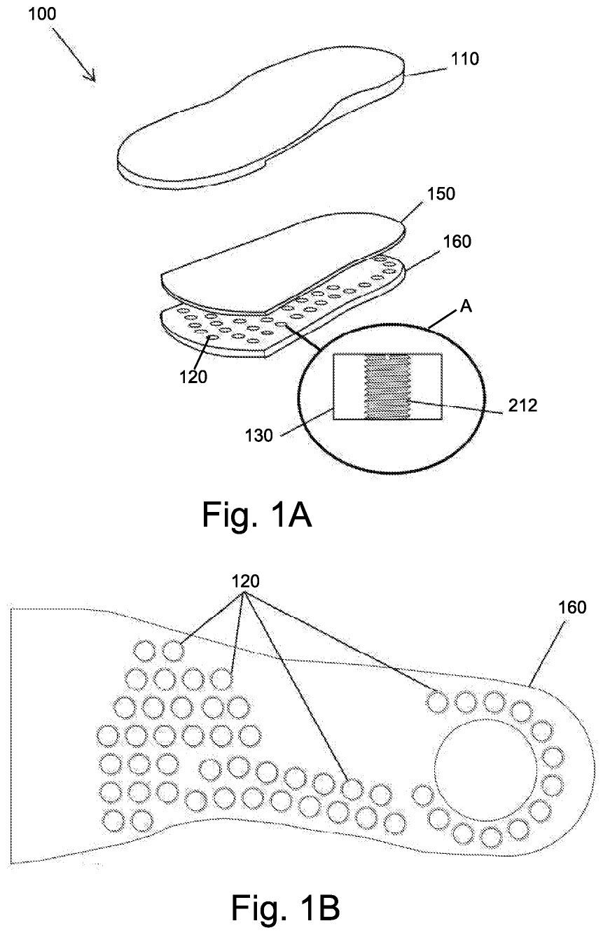

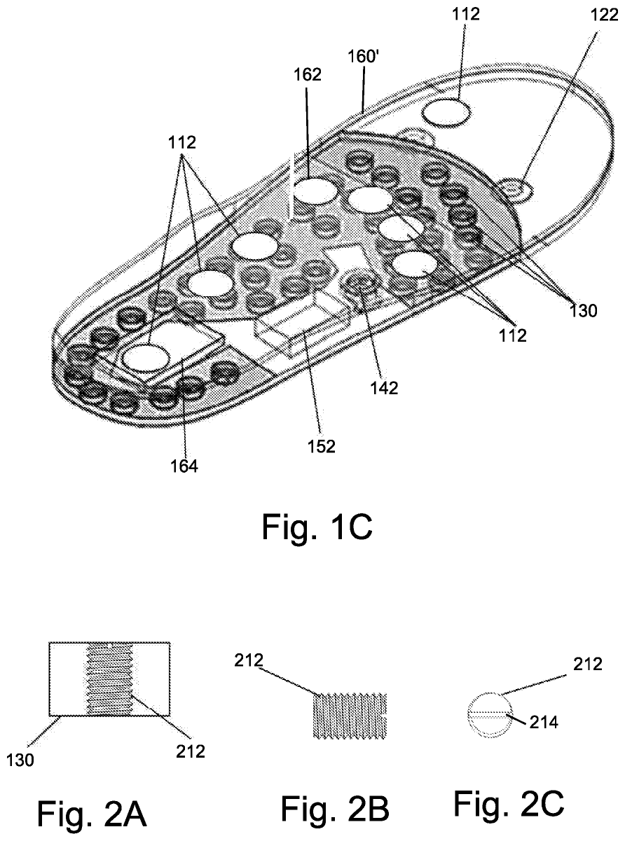

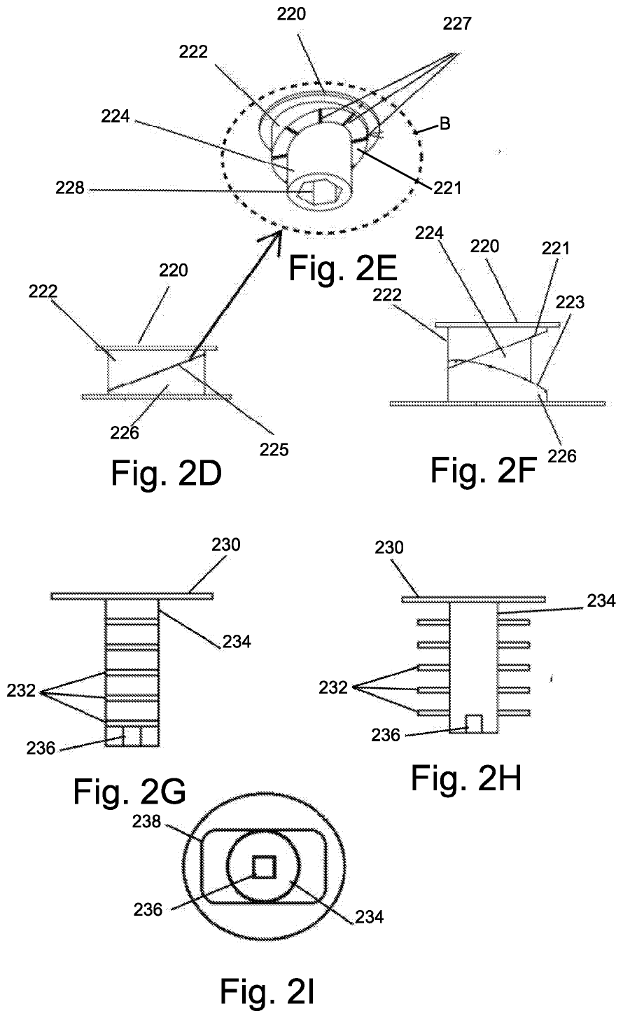

FIG. 1A is an exploded view, indicated generally as 100, of layers of an insole illustrating incorporation of height adjustment mechanisms according to an exemplary embodiment of the invention. In FIG. 1A, 160 is a base plate layer, layer 150 is a flexible cover layer, and layer 110 is an upper insole layer. In some embodiments layer 160 is constructed of polypropylene. In some embodiments layer 150 is made from a thin plastic material that insulates adjustment mechanisms attached to base plate layer 160 from the upper insole layer 110 and/or improves comfort. According to various exemplary embodiments of the invention layer 110 is made from one of foam, gel, Polyurethanes, cloth, and/cloth-like material. Layer 160 comprises a plurality of holes 120, each of which is fitted with a height adjustment mechanism 130 (shown in inset A) described below in the context of FIG. 2A. In the embodiment shown in inset A, height adjustment mechanism 130 is a screw 212 that passes through a hole 120 in base plate layer 160. In some embodiments each height adjustment mechanism 130 includes a manual adjustment interface. According to various exemplary embodiments of the invention each height adjustment mechanism 130 is independently selected from the group consisting of screws, a diagonal joint rotary lift, and a bayonet mount.

FIG. 1B schematically shows a top view of a base plate layer 160 of insole 100 according to an exemplary embodiment of the invention. The depicted embodiment comprises a plurality of holes 120 into which height adjustment mechanisms 130 (not shown in this figure) are installed. Holes 120 are distributed under the heel, under the arch and under the ball of the foot.

FIG. 1C is a top perspective view of an embodiment of a base plate layer 160′ of an insole 101 that comprises all features of insole 100 illustrated in FIGS. 1A and 1B and additional features to base plate layer 160. In this view, height adjustment mechanisms 130 are visible. In the depicted embodiment, base plate layer 160′ includes a magnetic connector 142 and battery 152. According to this embodiment battery 152 powers monitoring, analysis and connectivity capabilities and connector 142 facilitates connection to an external power source for charging. Magnetic connector 142 contributes to convenience of charging without taking insole 100′ out of the shoe. In some embodiments using magnetic charging cable(s) (not depicted) it is possible to charge insoles 100′ in both shoes with a dedicated cable. In other embodiments magnetic connector 142 can be moisture and/or waterproof. Other exemplary embodiments of the invention, employ wireless charging (e.g. with a dedicated charging mat upon which shoes containing the insoles are placed). In other exemplary embodiments of the invention, a kinetic energy charging system is embedded in the insole.

According to various exemplary embodiments of the invention battery 152 provides power to one or more of: sensors (e. g. 112 and/or 162), micro-processors 164, inertial measurement units (IMUs), System on Chip (SoC); board memory; connectivity modules; and stimulation hardware (e.g., vibration motor, haptic engine, and mini piezoelectric actuators). In some embodiments the microprocessor 164 in insole 100′ is configured to record data in an “offline mode” for a few hours or other period of time without connectivity to a smart device. According to these embodiments battery 152 provide power to operate monitoring, diagnostic and vibration capabilities. In the depicted embodiment, vibration motor 122 applies low-level vibration, which is known to contribute to improvement in nerve function.

Improvement in nerve function has the potential to contribute to wellness and/or performance for many different types of wearers. For example, low-level vibration is potentially beneficial for stroke- or diabetes-related nerve damage and/or to improve balance and/or improve gait. Low level vibration might also be useful for one or more of the following: to prevent injuries, falls, and to help to assess nerve damage and decrease of plantar sensation. In some exemplary embodiments of the invention, delivering imperceptible mechanical vibrations to the feet, enhancing nerve sensory performance—which in turn improves one's balance, gait control, and sense of the spatial position and movement of different parts of the body. In some embodiments operation of vibration motor 122 is activated via a signal transmitted from a smartphone or by a third party connected device or a remote care management platform. In the depicted embodiment, pressure sensors 112 are positioned in key points where ulceration and/or high pressure are most often observed. In the depicted embodiment, temperature sensors 162 (only 1 is depicted for clarity but 3, 5, 7, 9 or intermediate or greater numbers are present in other embodiments) are positioned in one or more areas that statistically and medically are at high risk for ulceration and other pathologies.

FIG. 2A is a transverse cross section of an exemplary screw based height adjustment mechanism 130 according to some exemplary embodiments of the invention. In the depicted embodiment base plate layer 160′ (see FIG. 1C) contains a screw 212. FIG. 2B is a side view screw 212. FIG. 2C is a bottom view of screw 212 showing a manual adjustment interface for rotating screw 212. In this embodiment the manual adjustment interface has the form of a screwdriver slot 214. Manual rotation of slot 214 with a screwdriver causes screw 212 to ascend or descend within an internally threaded hole 120 in base 160′.

FIG. 2D is a side view of an exemplary diagonal joint rotary lift height adjustment mechanism according to some exemplary embodiments of the invention in a closed (retracted) operational state. FIG. 2E is an inset depicting an upper portion of the mechanism disengaged from the lower portion in a bottom perspective view. According to the depicted embodiment, rotation of shaft 224 causes a change in orientation of upper cover portion 222 with respect to lower cover portion 226 along contact surface 225. As a result, top surface 220 is raised as depicted in FIG. 2F. Rotation of shaft 224 in the opposite direction lowers top surface 220.

Referring now to the inset B of FIG. 2E, extension (or retraction) of shaft 224 is achieved by rotation of manual adjustment interface 228 which is depicted here as a hexagonal socket that can be turned by an Allen wrench. Rotation of interface 228 moves teeth 227 on a bottom side 221 of contact surface 225 of upper portion 222 with respect to corresponding teeth (not depicted) on a top side 223 (FIG. 10F) of contact surface 225 of lower portion 226. Engagement of corresponding sets of teeth at 221 and 223 prevents rotation of 222 with respect to 226 in the absence of rotational force provided via manual adjustment interface 228.

FIG. 2G is a side view of an exemplary bayonet mount height adjustment mechanism 230 according to some exemplary embodiments of the invention. FIG. 2H is a side view of the bayonet mount height adjustment mechanism 230 of FIG. 2G rotated 90° with respect to FIG. 2G.

FIG. 2I is a bottom view of a portion of the exemplary height adjustment mechanism of FIGS. 2G and 2H showing a manual adjustment interface, in the depicted embodiment insertion of a square rod in square socket 236 in the bottom of shaft 234 allows rotation of shaft 234 with respect to rectangular hole 238 in base plate layer 160 (FIG. 1A) or 160′ (FIG. 1C).

In the depicted embodiment, shaft 234 is fitted with rectangular protrusions 232. When these protrusions 232 (FIG. 2H) are aligned with rectangular hole 238 (FIG. 2I), shaft 234 is free to ascend/descend through hole 238. Rotation of shaft 234 in either direction causes protrusions 232 to be misaligned with rectangular hole 238 and prevents shaft 234 from ascending/descending through hole 238. In this way rotation of square socket 236 unlocks the mechanism every 90° and relocks it as soon as protrusions 232 become unaligned with hole 238.

In the above described embodiments of insole 100 the base plate layers 160, 160′ are manufactured with an array of height adjustment mechanisms 130 located at specific locations. In the case of base plate layers 160′, sensors occupy space that cannot be allocated to height adjustment mechanisms resulting in areas of the foot that cannot be adequately treated.

In the following will be described embodiments of insoles in which the base layer comprises an array of holes covering the heel, arch, and ball of the foot. Height adjustment components can be inserted into and locked in place in any one of these holes in order to raise or lower the height of the insole at the location of the hole to help alleviate a problem, e.g. excessive pressure or temperature, at any specific location. Locations requiring height adjustment can be determined using various sensors that are located on a flexible PCB board that is positioned as a sensor level above the base layer of the insole.

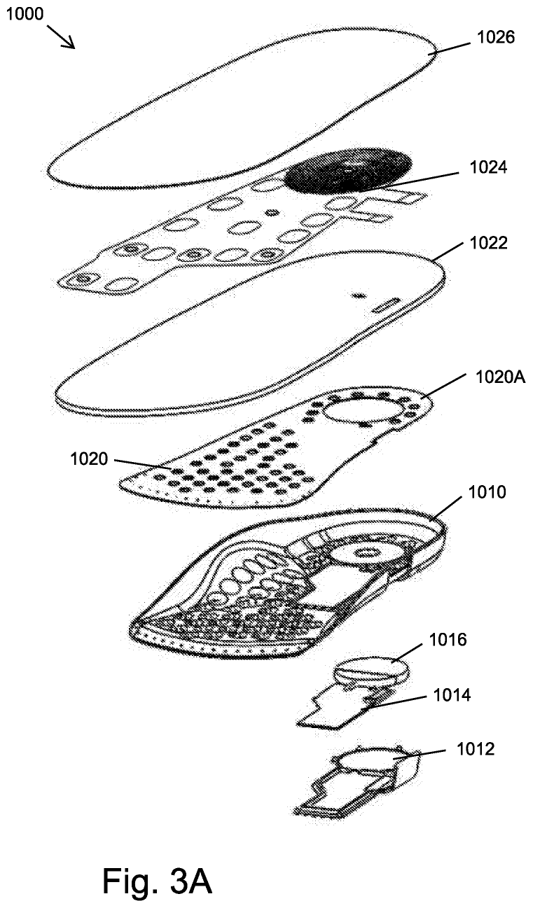

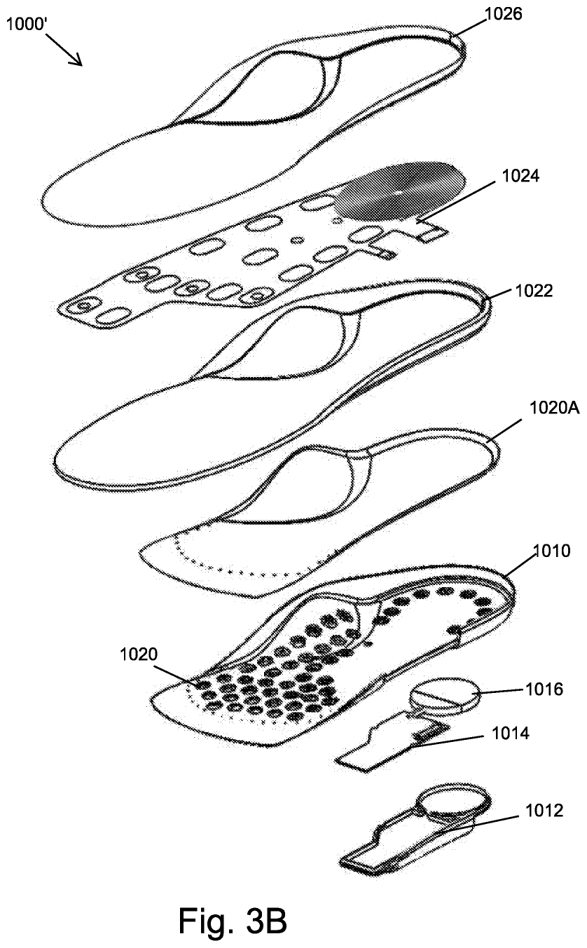

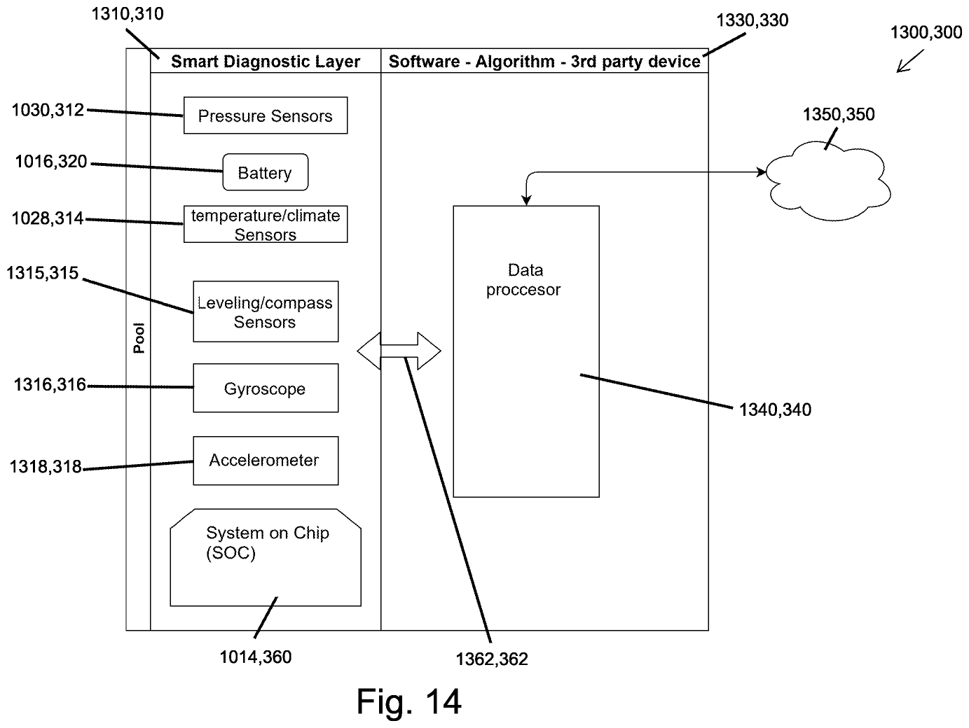

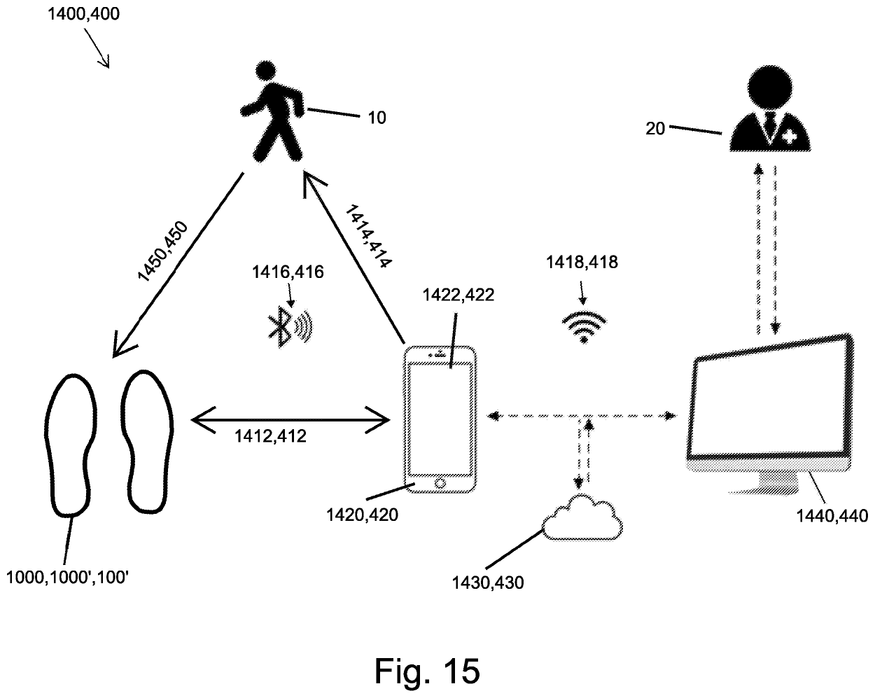

FIG. 3A is a schematic exploded view of an embodiment of an insole 1000 according to these embodiments. Insole comprises several layers starting with base layer 1010. Base layer 1010 is made of a rigid material, e.g. polypropylene. It comprises an array of holes 1020, wherein the holes pass through the base layer from its bottom to its top. A corresponding array of holes 1020 is shown in distribution layer 1020A. Layer 1020A is a distribution layer made of a flexible material, e.g. polypropylene, that is designed to distribute forces across the area of the insole so as to eliminate sharp points under the foot. This top view of base layer 1010 has a clear area corresponding to the shape of a socket in the bottom side of the base layer into which electronics 1014, which will be described herein below with reference to FIGS. 14 and 15; battery 1016; and magnet 1018 used to align the a charger for wireless charging with the electronics are inserted. After these components are fitted into the socket they are secured in place by lid 1012.

Above distribution layer 1020A is located a foam layer 1022 to insulate a flexible sensor layer 1024 from the height adjustment components attached to the base layer 1010 and to improve comfort of the user. Finally a fabric layer 1026 is at the top of insole 1000, forming a smooth interface with the bottom of the user's foot.

FIG. 3B symbolically shows another embodiment of an insole 1000′. Insole 1000′ is essentially identical to insole 1000, with the exception of distribution layer 1020A′, which does not comprise holes 1020. The reason for the difference between distribution layers 1020A and 1020A′, which are related to the type of height adjustment component used, will be described herein below.

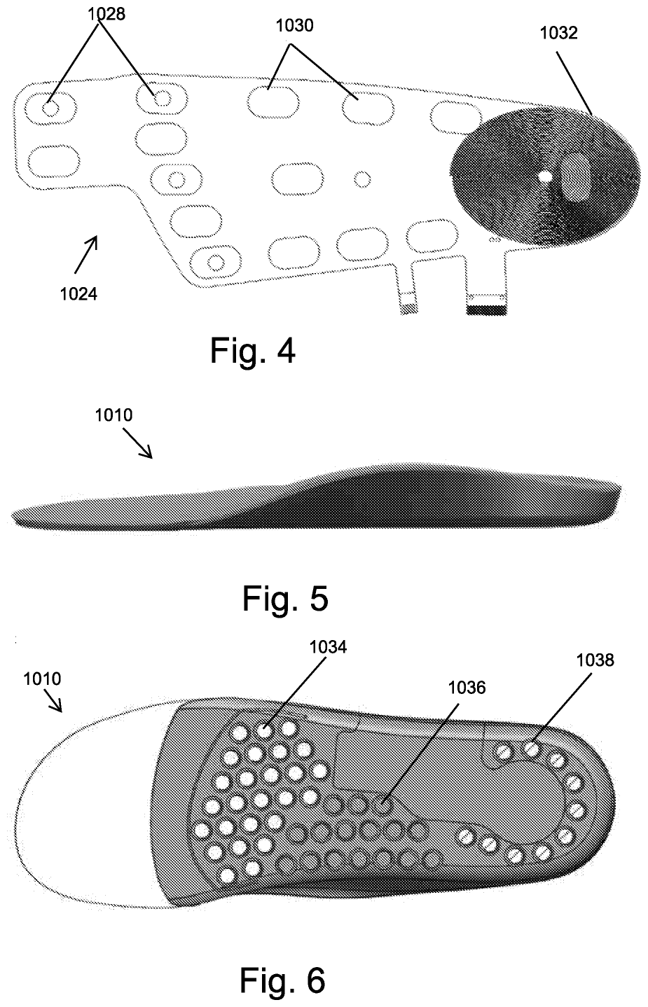

FIG. 4 schematically shows an exemplary embodiment of a sensor layer 1024 in the insoles 1000 and 1000′ shown in FIGS. 3A and 3B. Sensor layer 1024 is comprised of a flexible PCB. In the embodiment shown oval shapes symbolically represent pressure sensors 1030 and circles symbolically represent, temperature sensors 1028, oval shapes with empty centers symbolically. An antenna 1032 for charging the battery 1016 is located at the heel end of the sensor layer. In other embodiments other types and distributions of sensors can be present on sensor layer 1024 (see, for example, FIG. 14). In some embodiments, pressure sensors 1030 and temperature sensors 1032 and/or antenna coil 1032 are all printed to create flexible PCB.

In some embodiments, sensor layer 1024 layout could be different in location of the sensors and/or the flexible PCB shape.

FIG. 5 schematically shows the three dimensional shape of base layer 1010. This shape conforms to the contour of a human foot and is provided in many different dimensions to fit different shoe sizes and to provide a starting treatment point for a user suffering from a particular condition.

FIG. 6 is a bottom view of the base layer 1010 showing an exemplary array of holes that pass from the bottom through to the top of layer 1010. In order to enable providing exact instructions for raising or lowering the height of the insole at specific locations, the holes 1020 are identified as members of three groups: group 1034 for the holes under the ball of the foot (circles with white centers), group 1036 under the arch (circles with grey centers), and group 1038 under the heel (circles with white centers and a line through them). Beside each hole in each group is printed an identifying number (not shown in the figures for clarity).

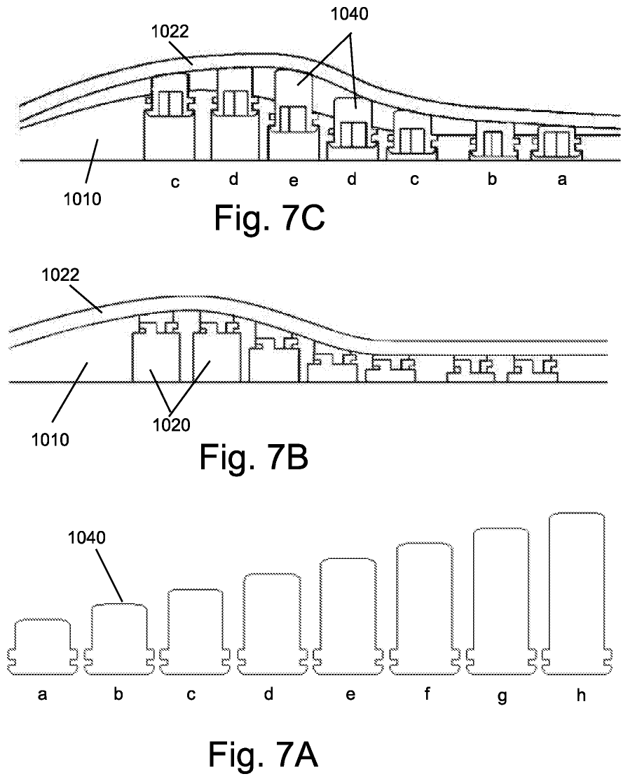

FIG. 7A shows a set of height adjustment components in the form of rods 1040 that are used in insole 1000′. In this embodiment the set comprises 8 rods of gradually increasing heights identified with the letters a-h. In the exemplary embodiment shown, the heights of the rods increase from 6 mm to 20 mm in even steps of 2 mm. In exemplary embodiment typical diameters of the rods are 6 mm or 8 mm. In other embodiments, the number of rods and their heights and/or diameters can be more or less than those in this example. The height adjustment rods 1040 are firmly attached to the base layer 1010 by a bayonet connection comprised of pins 1070 located near the bottom of each rod 1040 that fit into slots 1072 near the top of each hole 1020 that passes through the rigid material of the base layer 1010. In order to insert or remove a rod 1040 from a hole 1020, a tool, e.g. a screw driver or Allen torque wrench, is fitted into a corresponding slot or hexagonal socket on the bottom of each rod 1040 allowing the rod 1040 to be rotated clockwise or counterclockwise to engage or disengage the pins 1070 on the rod 1040 from the slots 1072 in the hole 1020.

FIG. 7B symbolically shows a number of holes 1020 in an insole 1000 without any height adjustment components installed in them. In this configuration distribution layer 1020A′ lies on the top surface of rigid base layer 1010 and distribution layer 1020A′ and the layers above it assume the contour of the base layer.

FIG. 7C symbolically shows the holes 1020 in FIG. 7B with height adjustment rods 1040 inserted in them. In this case the distribution layer 1020A′ is moved up at varying distances from the top surface of rigid base layer 1010 changing the surface contour of all layers of insole 1000′ above base layer 1010. The letters under the vertical arrows at the bottom of each hole correspond to the reference letters identifying the rods 1040 in FIG. 7A.

Instead of the embodiment of the insole 1000′ in which the height adjustment components are rods 1040, the height adjustment components in insole 1000 comprise disks 1042 that lock into the holes 1020 in the rigid base layer 1010. The disks can be stacked on top of each other to provide height adjustment components of the appropriate height to obtain a desired contour of the surface of the distribution layer 1020A.

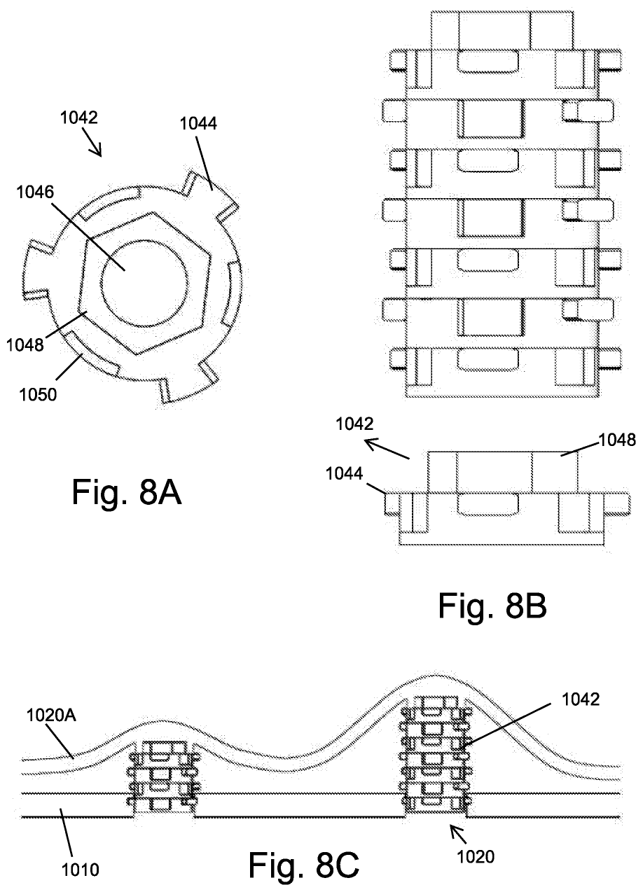

FIG. 8A schematically shows a top view of an embodiment of a disk 1042 that will form a component of a height adjustment component in the insole 1000 of FIG. 3. Seen in the figure are the three disk lock teeth 1044, which are used to lock the disk 1042 to the base layer 1010 of the insole; three recesses 1050 in the side of the disk 1042 designed to mate with a special tool designed to add a disk to ones below it or to release it, thereby increasing or decreasing the height of the stack; a nipple 1048, having a hexagonal cross section, which “clicks” into a socket having a hexagonal cross section in the bottom of disk 1042 like Lego© bricks to provide a height adjustment component when two or more disks 1042 are stacked on top of each other, and hole 1046 that passes through disk 1042. The function of the hole 1046 will be described herein below. FIG. 8B shows a side view of a single disk below and a stack of seven disks above it. FIG. 8C shows the influence of height adjustment components comprised of stacks of different numbers of disks 1042 located in holes 1020 in base layer 1010 on the contour of the distribution layer 1020A of insole 1000. It is noted that in the embodiment shown in the figures the disk nipple 1048 and socket on the bottom of the disks 1042 have hexagon cross sections; however in other embodiments the cross sections can have other polygon shapes. Also in embodiments disk 1042 can have more or less than three lock teeth 1044. Exemplary dimensions of the disks 1042 are 6 mm or 8 mm diameter and thickness (height) 2 mm.

As the height of the stack of disks 1040 grows a small amount of wobble appears which can cause instability of the contour of the upper surface of insole 1000. For this reason distribution layer 1020A comprises holes 1020 into which disk nipple 1048 of the top disk 1042 in the stack fits to provide stability to the height adjustment components. In addition the holes 1020 in distribution layer 1020A are needed to anchor a special tool that is used to insert or remove disks 1042 that form the height adjustment components of insert 1000.

Holes are not needed in distribution layer of insole 1000′, since the bayonet connection that holds rods 1040 in the holes 1020 insures that each height adjustment rod 1040 remains vertical insuring that the contour of the upper surface of insole 1000′ remains exactly as required.

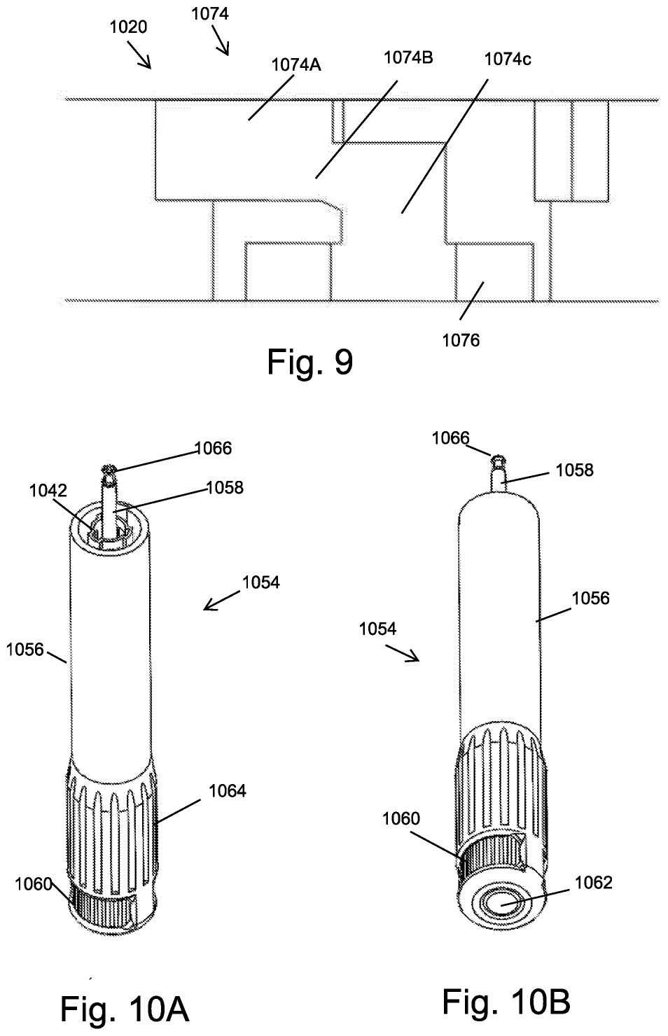

FIG. 9 schematically shows a cross-sectional view the upper part of an empty hole 1020 in base layer 1010 that has been configured for the insertion of disks 1042. When a disk 1042 is inserted from the bottom of base layer 1010 using a tool 1054 (see FIGS. 10A to 10C) the lock teeth 1044 align with channels 1074, which comprise two vertical sections 1074A and 1074C and one horizontal section 174B that have been created in the walls of each hole 1020. As the disk 1042 is pushed upwards it travels until the lock teeth 1044 hit the top of channel section 1074C. At this point the disk is turned 30 degrees clockwise as seen from the bottom of base layer 1010. This rotates the lock teeth 1044 along the channel section 1074B until they are stopped at the end of this channel section. At this point, the lock teeth have exited the insertion vertical section of channel 1074 and are now confined in the horizontal channel section, thereby locking the disk 1042 to the base layer 1010 of insole 1000. If it is required to add a disk to the stack, then turning the disk 1042 a further 30 degrees clockwise (as will be explained herein below, by inserting another disk from the bottom as a repeat of the above instructions) aligns the first disk 1042 with vertical channel section 1074A so that it can exit the base layer 1010 and be held in place in the stack by the second disk which is now in locked to the base layer 1010 with its lock teeth 1044 in horizontal section 1074B of channel 1074 Correct in the sides of the hole 1020. This process of insertion of disks continues building up the disk stack until the required height is attained. Removal of disks follows the above steps in reverse with a counterclockwise turn at each step.

FIGS. 10A, 10B, and 10C schematically show a dedicated tool 1054 configured to insert and remove disks 1042 through a hole 1020 in base 1010 of insole 1000 to build a height adjustment component composed of a stack of disks 1042 or to remove disks 1042 to reduce the height of the stack.

Tool 1054 is comprised of an elongated cylindrical barrel 1056, which is contains in its interior a supply of disks 1042. On the base of barrel 1056 is a lock button 1062. Near the bottom of barrel 1056 is dial 1060 above which are optional ridges 1064 that provide a grip for holding and rotating the tool 1054. Inside barrel 1056 is a lock shaft 1058 that passes through holes 1046 in the disks 1042. Lock shaft 1058 is topped with an expansion element 1066 that is activated by activation shaft 1068, which passes through the interiors of the lock shaft 1058 and the expansion element 1066.

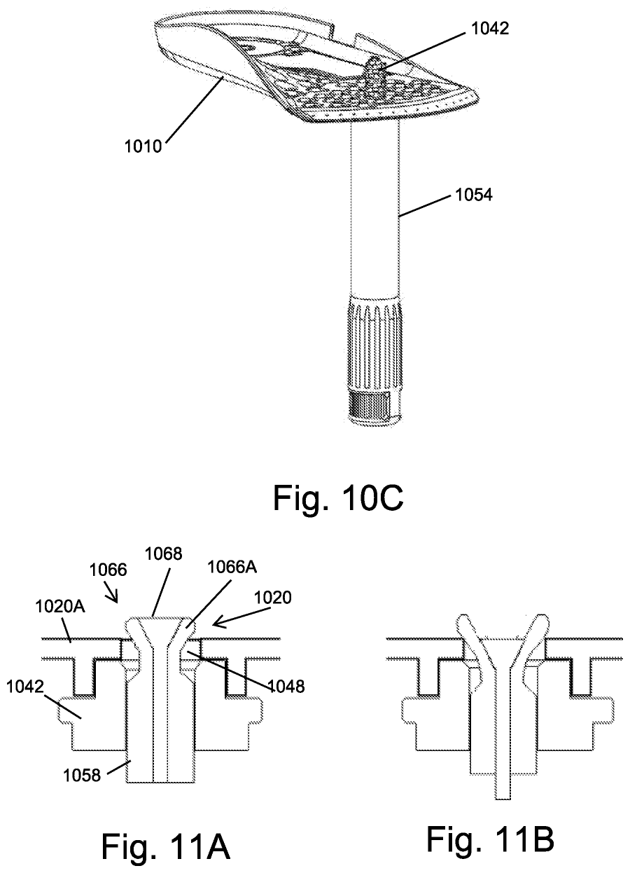

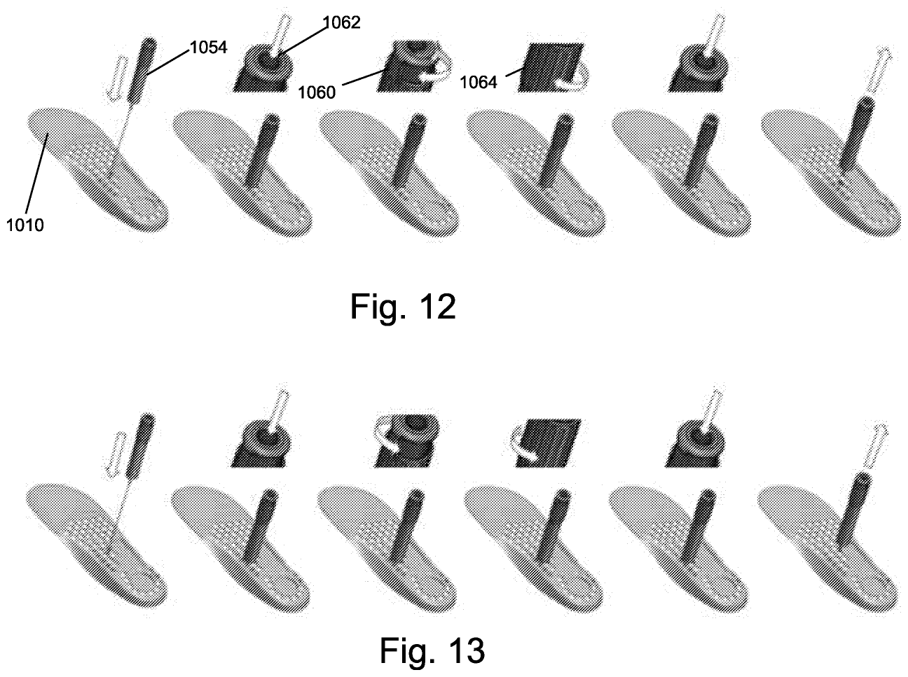

FIGS. 11A and 11B are cross sectional views that schematically show respectively how an expansion element of a tool of FIGS. 10A and 10B is locked and unlocked in a hole of the distribution layer 1020A of the insole of FIG. 3A The expansion element 1066 has the shape of a funnel that has been divided into sections 1066A (in this exemplary embodiment three sections). Activation shaft 1068 passes through the center of lock shaft 1058 to expansion element 1066. The end of activation shaft 1068 that is inside of expansion element 1066 has conical shape so that pulling downwards on activation shaft 1068 forces the sections 1066A of expansion element 1066 apart as shown in FIG. 11B until the diameter of expansion element 1066 is greater than the diameter of the hole distribution layer 1020A and the sides of the sections 1066A of the expansion element 1066 press against the distribution layer 1020A with enough force to lock the tool 1054 to the distribution layer 1020A. Pushing up on activation shaft 1026 allows the sections of expansion element 1066 to move together reducing the diameter of expansion element 1066 until it is less than the diameter of the hole 1046 in disk 1042, thereby unlocking the tool 1054 from the disk 1042 as shown in FIG. 11A. Note that when the expansion element 1066 is in hole 1020, the nipple of the top disk 1042 in the stack of disks is inserted into hole 1020 and will remain therein when the tool 1054 is removed in order to stabilize the stack of disks.

FIG. 12 shows the process for using tool 1054 for adding disks 1042 to a stack. The steps of the process are:
1. Locate the tool 1054 at the hole 1020 required on the underside of the base layer 1010 of the insole 1000.
2. Lock the tool 1054 in place by inserting the lock shaft 1058 through hole 1020 in base layer 1010 and expansion element 1066 fully into hole 1020 in distribution layer 1020A and pressing lock button 1062 on tool 1054. When lock button 1062 is pressed, activation shaft 1068 is pulled downwards forcing its conical end into the center of expansion element 1066 spreading the sections of expansion element 1066 locking tool to distribution layer 1020A.
3. Prepare one disk 1042 by turning the dial 1060 of the tool 1054 clockwise one click. This advances the top disk of the disks in the barrel 1056, readying it to be inserted into the first vertical channel 1074C in the hole 1020.
4. Turn the tool clockwise until the disk 1042 prepared in step 3 clicks into place. When tool 1054 is rotated, the disk 1042 being inserted rotates the disk which is already locked in place, thereby unlocking the “old” disk from the base layer 1010 and allowing it to move upward to make space for the disk being inserted, which becomes locked into the hole. As the disc moves up the nipple 1048, of the newly inserted disk “clicks” into the socket in the bottom of the “old” disk.
5. Repeat steps 3 and 4 as often as necessary to add more disks 1042.
6. Push lock button 1062 to release the tool 1054 from the distribution layer 1020A of the insole 1000, leaving the last disk 1042 added in place. When the lock button is pressed a second time, activation shaft 1068 is pushed upwards allowing the sections of expansion element 1066 to close, thereby releasing the tool 1054 from hole 1020 in distribution layer 1020A and allowing the tool to be withdrawn.

FIG. 13 shows the process for using tool 1054 for removing disks 1042 from a stack. The steps of the process are:
1. Locate the tool at the point required on the underside of the insole;
2. Lock the tool 1054 in place by inserting the lock shaft 1058 through hole 1020 in base layer 1010 and expansion element 1066 fully into hole 1020 in distribution layer 1020A and pressing lock button 1062 on tool 1054. When lock button 1062 is pressed, activation shaft 1068 is pulled downwards forcing its conical end into the center of expansion element 1066 spreading the sections of expansion element 1066 locking tool to distribution layer 1020A.
3. Prepare one disk by turning the dial 1060 of the tool 1054 counter-clockwise one click. Turning the dial 1060 of the tool 1054 counter-clockwise moves the disks 1042 in the barrel 1056 or the tool 1054 down making space in the barrel 1056 for one disk to be removed from the hole 1020 in the base level 1010 of the insole 1000.
4. Turn the tool counter-clockwise until one disk clicks out of the hole 1020 in the base level 1010 of the insole 1000. When tool 1054 is turned counter-clockwise, as the disc moves down the nipple 1048 of the disk “clicks” out of the socket in the bottom of the disk above it and the disk 1042 being removed rotates the disk above it, thereby locking that disk above the disk being removed to the base layer 1010.
5. Repeat steps 3 and 4 to remove more disks as necessary.
6. Push the button to release the tool from the insole.

As described herein above, embodiments of the insole, e.g. insole 100′, 1000, and 1000′ schematically shown in FIG. 1C, FIG. 3A, and FIG. 3B, include one or more sensors. FIG. 14 is a schematic representation of a data acquisition layer, indicated generally as 300,1300 of an insole 100′,1000,1000′ according to some exemplary embodiments. In the following description of FIG. 14, reference numerals 400-450 refer to insole 100′ and reference numerals 1400-1450 refer to insoles 1000 and 1000′.

According to various exemplary embodiments the sensors include one or more of: pressure sensors 312,1030; sensors 314,1028 to obtain foot temperature microclimate temperature and humidity data of the foot in the insole of the footwear; leveling/compass sensors 315,1315; gyroscopes 316,1316; and accelerometers 318,1318. The group of sensors of various types is indicated in FIG. 14 as smart diagnostic layer 310,1310. In the depicted embodiment, layer 310,1310 also includes a battery 320,1016 and system on chip (SOC) 360 or electronics 1014. In some embodiments SOC 360 or electronics 1014 includes integrated Wi-Fi and Bluetooth modules. In the depicted embodiment, data acquisition layer 300,1300 includes a data communication channel 362,1362 configured to transmit an output signal from the one or more sensors to a data processor 340,1340. In the depicted embodiment, data processor 340,1340 is equipped with a software algorithm 330,1330 and/or is in communication with analytic software via network 350,1350. In some exemplary embodiments, the analytic software is cloud based. In some exemplary embodiments, data processor 340,1340 is physically present in insole 100′,1000,1000′. In other embodiments data processor 340,1340 is physically present in an external device.

FIG. 15 is a schematic representation of exemplary embodiments of a system indicated generally as 400,1400 which assists a user in adjusting any of the insoles described herein above. Systems 400 and 1400 can be generally described respectively as a data processor 420,1420 designed and configured to receive one or more output signal(s) 412,1412 from sensors in insoles 100′,1000,1000′ and translate the output signal(s) to user advice 414,1414 and a user interface 422,1422 presenting user advice 414,1422 to user 10.

In the following description of FIG. 15, reference numerals 300-362 refer to insoles 100′ and reference numerals 1300-1362 refer to insoles 1000 and 1000′.

The embodiment of system 400 includes one or more insoles 100′ with a plurality of height adjustment mechanisms integrated across at least a portion of an area thereof and a manual adjustment interface for each of the height adjustment mechanisms 130. Exemplary height adjustment mechanism types and manual adjustment interfaces are described herein above. In addition, insoles 100′ include one or more sensors providing one or more output signal(s) 412. System 1400 includes one or more insoles 1000,1000′ comprising a base layer 1010 containing an array of holes 1020 into which height adjustment components comprised of rods 1040 (insole 1000′) or disks 1042 (insole 1000) can be inserted either by the wearer of the shoe depicted as user 10 in the systems 400,1400 of FIG. 15 or by a health care professional 20.

In the depicted embodiment, systems 400, 1400 includes a data processor 420,1420 (depicted here as a smartphone) designed and configured to translate output signal(s) 412,1412 to alert and/or user advice 414,1414 and a user interface 422,1422 (depicted here as the screen of the smartphone) presenting user advice 414,1414. In some embodiments user advice 414,1414 results from a diagnosis process that wirelessly connects 418,1418 to cloud network 430,1430 to compare scientific data and/or save data and/or run artificial intelligence (AI) and machine learning (ML) algorithms.

In system 400 user 10 receives alert and/or user advice 414 from interface 422 and performs manual adjustment 450 of each of the plurality of height adjustment mechanisms 130 in accordance with received advice 414. In system 1400 user 10 receives alert and/or user advice 1414 from interface 1422 and inserts, removes or changes the height of the height adjustment component in designated holes 1020 in accordance with received advice 1414.

In some embodiments user advise 422,1422 is generated by software algorithms in data processor 420,1420, which is configures to allow the user 10 to monitor and manage his/her feet health. In some embodiments, a specialist health care provider 20 can get and/or send alert and/or adjustment instructions and/or request the patient to visit in clinic for pressure offloading treatment 414, 1414 to user 10 remotely through interface 422. In both scenarios, the basis for user advise 422,1422 is data generated from output signals of the sensors in base plate layer 160′ in insole 100′ or on sensor layer 1020 in insoles 1000 and 1000′.

In the depicted embodiment, data processor 420,1420 resides in a device external to insoles 100′,1000, 1000′. According to various exemplary embodiments of the invention the device external to the insole is a smartphone (as depicted), a PC (personal computer), a smartwatch or a tablet. In some embodiments the system includes a wireless communication link (e.g., Bluetooth 416,1416) between the insole 100′,1000,1000′ and data processor 420,1420.

In other exemplary embodiments, data processor 420 resides in insoles 100′,1000,1000′. According to these embodiments, user advice 414,1414 is transmitted directly from insoles 100′,1000,1000′ and presented to user 10 via interface 422,1422, which is an external device, e.g. a smartphone.

In some embodiments the sensors are always gathering data and completing a user's gait analysis and/or pressure distribution map and/or temperature map which is transferred to user interface 422,1422 of patient 10, or interface 440,1440 of health specialist 20, or to both. Using different algorithms and ML, current information can be cross-referenced and various pathological patterns identified over time and therefore to generate user advice 414,1414.

In some embodiments a health professional 20 can adjust each height adjustment mechanism 130 manually or insert or remove height adjustment components (rods or disks) in specific holes 1020 according to his knowledge and physical examination. A health professional 20 or user 10 can decide according to the diagnosis (e.g. pressure map) shown in the user interface where to adjust the height by seeing the high pressure and/or temperature areas on map and intuitively act by the software recommendation (e.g. “High pressure detected in medial left arch. Change x1y1, x2y2 high adjustment mechanisms in +2 mm) or remotely, done by user 10 but guided by the health specialist 20. The health professional 20 can also send a clinic invite directly to the patient 10 for a further observation and/or treatment and/or adjustment.

Systems 400,1400 can perform continuous monitoring of foot plantar pressure and/or temperature and/or user's gait which is used to diagnose early foot ulceration forming and as result to dynamically output alerts and/or medical advice to user (offloading treating plan using the adjustments mechanisms) 414,1414 to user interface 422,1422 and/or to health specialist 20 via interface 440,1440. Consequently systems 400,1400 can lead to reduction in diabetic foot ulcer recurrence. In some embodiments user advice 414,1414 includes an alert concerning a nascent foot ulcer. In some embodiments the alert includes offloading adjustments instructions when aberrant pressures were detected and/or correcting user's gait and/or orthopedic disorder.

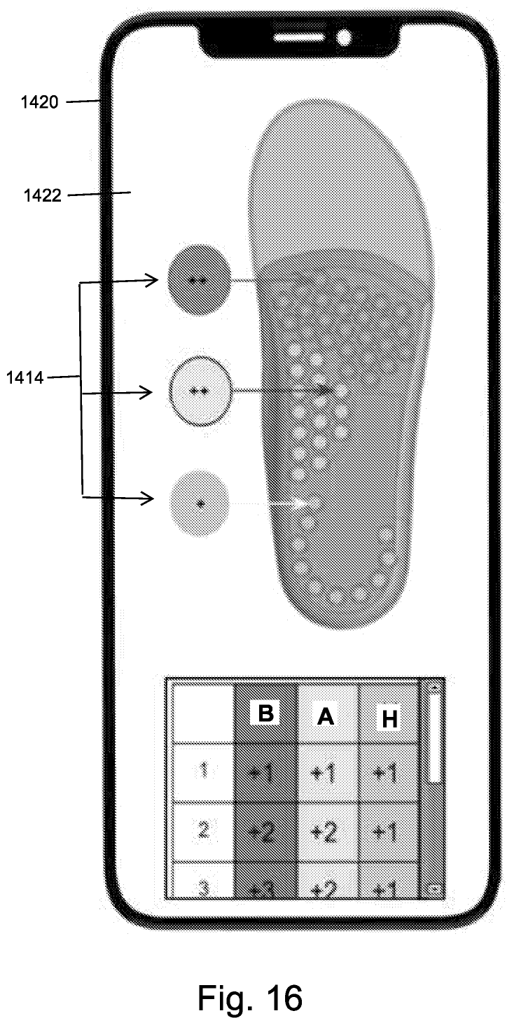

FIG. 16 shows an exemplary screen shot of the user advice 1414 on interface 1422 of data processor 1420 for an embodiment of insert 1000 in which the height adjustment components are disks 1020.

At the top of the screen is an image of the bottom of the base layer 1010 showing the array of holes 1020. Three specific holes are pointed out and the table at the bottom of the screen indicates how many disks should be added to each hole in the ball B, arch A, and heel H areas of the insole.

Although embodiments of the invention have been described by way of illustration, it will be understood that the invention may be carried out with many variations, modifications, and adaptations, without exceeding the scope of the claims.

1. An insole comprised of the following layers:
a) a base layer comprising an array of holes that pass through the base layer, the base layer located at the bottom of the insole;
b) a distribution layer that is designed to distribute forces across the area of the insole, the distribution layer located above the base layer;
c) a foam layer, the foam layer located above the distribution layer;
d) a sensor layer, the sensor layer located above the foam layer; and
e) a fabric layer, the fabric layer located at the top of insole.

2. The insole of claim 1 comprising height adjustment components configured to be inserted into and locked in place in any one of the holes in the base layer, wherein the height adjustment components are configured to raise or lower the height of the insole at the location of the hole.

3. The insole of claim 1 wherein, the bottom side of the base layer comprises a socket into which electronics and a battery are inserted.

4. The insole of claim 1 wherein, the distribution layer comprises an array of holes that pass through the distribution layer and that corresponds to the array of holes in the base layer.

5. The insole of claim 1 wherein, locations requiring height adjustment are determined using various sensors that are located on the sensor layer.

6. The insole of claim 1 wherein, the sensor layer is a flexible printed circuit board that comprises at least one of each of at least one of the following: pressure sensors, temperature sensors; humidity sensors; leveling sensors; compass sensor; gyroscopes; accelerometers; and antennas.

7. The insole of claim 1 wherein, the height adjustment components are rods of varying heights.

8. The insole of claim 7 wherein, the height adjustment rods are firmly attached to the base layer by a bayonet connection comprised of pins located near the bottom of each rod that are configured to fit into slots near the top of each hole that passes through rigid material of the base layer.

9. The insole of claim 4 wherein, the holes in the base layer of the insole comprise at least one channel composed of two vertical sections and one horizontal section created in the walls of the hole; wherein, the first vertical section has a first end at the bottom of the insole and a second end ending at a first end of the horizontal section and the second vertical section has a first end beginning at a second end of the horizontal section and ending at the top of the insole.

10. The insole of claim 9 wherein, the height adjustment components are composed of at least one disk comprising: at least one disk lock tooth configured to lock the disk to the base layer of the insole; recesses configured to mate with a tool designed to add the disk to a disk below it or to release the disk from a disk above it; a nipple having a polygon cross section at the top of the disk; a socket having a hexagonal cross section in the bottom of the disk; and a hole passing through the disk.

11. The insole of claim 10 wherein, the disk is configured to be locked to the base layer by inserting the disk from the bottom of the insole with the nipple upwards and the lock teeth aligned with the first end of the first vertical sections of the channels, pushing the disk upwards until the lock teeth reach the second end of the first vertical section of the channel, and rotating the disk clockwise until the lock teeth enter the horizontal section of the channels.

12. The insole of claim 11 wherein, the disk is configured to be unlocked and removed from the base layer in one of two ways: i) by rotating the disk counterclockwise until the lock teeth reach the first end of the horizontal section of the channel; pulling the disk downward in the first vertical section of the channel until the lock teeth exit the first vertical section of the channel and removing the disk from the bottom of the base layer; and ii) by rotating the disk clockwise until the lock teeth reach the second end of the horizontal section of the channel; pushing the disk upward in the second vertical section of the channel until the lock teeth exit the second vertical section of the channel and removing the disk from the top of the base layer.

13. The insole of claim 10 wherein, the height adjustment component is a stack of at least two of the disks formed by inserting the nipple at the top of a bottom disk into the socket in the bottom of the disk above it and repeating the process adding as many disks as necessary to attain the required height.

14. A dedicated tool configured to insert a height adjustment component comprised of a disk or a stack of disks into the insole of claim 10.

15. The dedicated tool of claim 14, wherein the tool comprises: an elongated cylindrical barrel, which contains in its interior a stack of disks; a lock button located on the base of the barrel; a dial located near the bottom of the barrel; a lock shaft that passes through the holes in the disks; an expansion element attached at the top end of the lock shaft; and an activation shaft, which passes through the interiors of the lock shaft and the expansion element.

16. A method of using the dedicated tool of claim 14, to add at least one disk to a height adjustment component comprised of a disk or a stack of disks in the insole, the method comprising:
a) locating the tool at a required hole on the underside of the base layer of the insole;
b) locking the tool in place by inserting the lock shaft through the hole in the base layer and the expansion element fully into a corresponding hole in the distribution layer of the insole and pressing the lock button on the tool;
c) preparing one disk by turning the dial of the tool clockwise one click;
d) turning the tool clockwise until one disk clicks into place;
e) repeating steps c and d as often as necessary to add more disks; and
f) pushing the lock button on the tool to release the tool from the distribution layer and remove the tool from the insole leaving the last disk added in locked in the base layer.

17. A method of using the dedicated tool of claim 14, to remove at least one disk from a height adjustment component comprised of a disk or a stack of disks in the insole, the method comprising:
a) locating the tool at the point required on the underside of the insole;
b) locking the tool in place by inserting the shaft through the hole in the base layer and the expansion element fully into a corresponding hole in the distribution layer of the insole and pressing the lock button on the tool;
c) preparing one disk by turning the dial of the tool counter-clockwise one click;
d) turning the tool counter-clockwise until one disk clicks out of the hole in the base layer;
e) repeating steps c and d to remove more disks as necessary; and
f) pushing the button on the tool to release and remove the tool from the insole.

18. A system configured for adjusting the height of an insole at specific locations on the insole, the system comprising:
a) at least one insole according to claim 2;
b) a data processor configured to receive at least one output signal from sensors in the sensor layer of each insole and to translate the output signals to user advice;
d) a user interface on which the user advice is displayed;
wherein the user advice relates to adjustment of the height of at least one of the height adjustment components in each of the insoles.
